(19) United States
(12) Patent Application Publication
(10) Pub. No.: US 2023/0000376 A1
(43) Pub. Date: Jan. 5, 2023

(54) SYSTEM AND METHOD FOR PHYSIOLOGICAL MEASUREMENTS FROM OPTICAL DATA

(71) Applicant: BINAH.AI LTD, Tel Aviv (IL)

(72) Inventors: David MAMAN, Tel Aviv (IL); Konstantin GEDALIN, Tel Aviv (IL); Michael MARKZON, Tel Aviv (IL)

(21) Appl. No.: 17/780,988

(22) PCT Filed: Dec. 1, 2020
(86) PCT No.: PCT/IL2020/051238

§ 371 Date: May 29, 2022

Related U.S. Application Data
(60) Provisional application No. 62/942,247, filed on Dec. 2, 2019.

(51) Int. Cl. A61B 5/024; A61B 5/1455

(52) U.S. Cl. CPC A61B 5/02416; A61B 5/14551; A61B 5/021

(57) ABSTRACT

A new system and method is provided for improving the accuracy of pulse rate detection. Various aspects contribute to the greater accuracy, including but not limited to pre-processing of the camera output/input, extracting the pulsatile signal from the preprocessed camera signals, followed by post-filtering of the pulsatile signal. This improved information may then be used for such analysis as HRV determination, which is not possible with inaccurate methods for optical pulse rate detection.

### FIELD OF THE INVENTION

The present invention is of a system and method for physiological measurements as determined from optical data, and in particular, for such a system and method for determining such measurements from video data of a subject.

### BACKGROUND OF THE INVENTION

Heart rate measurement devices date back to 1870's with the first electrocardiogram (ECG or EKG), measuring the electric voltage changes due to heart cardiac cycle (or heart beat). The EKG signal is com-posed from three main components: P wave which represents the atria depolarization; the QRS complex represents ventricles depolarization; and T wave represents ventricles re-polarization.

A second pulse rate detection technique is optical measurement that detects blood volume changes in the microvascular bed of tissue named photo-plethysmography (PPG). In PPG measurement the peripheral pulse wave characteristically exhibits systolic and diastolic peaks. The systolic peak is a result of direct pressure wave traveling from the left ventricle to the periphery of the body, and the diastolic peak (or inflection) is a result of reflections of the pressure wave by arteries of the lower body.

There are two categories of PPG based devices: contact-based and remote (rPPG). The contact based device typically is used on the finger and measures the light reflection typically at red and IR (infrared) wave-lengths. On the other hand, the remote PPG device measures the light reflected from skin surface typically of the face. Most rPPG algorithms use RGB cameras, and do not use IR cameras.

The PPG signal comes from the light—biological tissue interaction, thus depends on (multiple) scattering, absorption, reflection, transmission and fluorescence. Different effects are important depending on the type of device, for contact based or remote PPG measurement. In rPPG analysis a convenient first order decomposition of the signal is to intensity fluctuations, scattering (which did not interact with biological tissues), and the pulsatile signal. The instantaneous pulse time is set from the R—time in EKG measurement or the systolic peak in a PPG measurements. The EKG notation is used to refer the systolic peak of the rPPG measurement as R time. The instantaneous heart rate is evaluated from the difference between successive R times, RR(n)=R(n)−R(n−1), as 60/RR(n) in beats per minutes.

Fluctuations in RR interval indicate how the cardiovascular system is adjusted to sudden physical and psychological challenges to homeostasis. The measure of these fluctuations is referred to as heart rate variability (HRV).

### BRIEF SUMMARY OF THE INVENTION

Accurate optical pulse rate detection unfortunately has suffered from various technical problems. The major difficulty is the low signal to noise achieved and therefore failure to detect the pulse rate. Accurate pulse rate detection is needed to be able to determine heart rate variability (HRV).

HRV is the extraction of statistical parameters from the pulse rate over a long duration. Traditionally the measured time varies from 0.5-24 hours, but in recent years researchers have extracted HRV also from substantial shorter time duration. The statistical information derived from the HRV may provide a general indicator of the subject's well being, including for example with regard to stress estimation.

The presently claimed invention overcomes these difficulties by providing a new system and method for improving the accuracy of pulse rate detection. Various aspects contribute to the greater accuracy, including but not limited to pre-processing of the camera output/input, extracting the pulsatile signal from the preprocessed camera signals, followed by post-filtering of the pulsatile signal. This improved information may then be used for such analysis as HRV determination, which is not possible with inaccurate methods for optical pulse rate detection.

The HRV parameters relate the status of the sympathetic nervous system (SNS) and parasympathetic nervous system (PNS) activities. The SNS and PNS are indicators for the individual stress level, allowing to estimate the stress index.

According to at least some embodiments, there is provided a method for obtaining a physiological signal from a subject, the method comprising obtaining optical data from a face of the subject with a camera, analyzing the optical data to select data related to the face of the subject with a computational device in communication with said camera, detecting optical data from a skin of the face, determining a time series from the optical data by collecting the optical data until an elapsed period of time has been reached and then calculating the time series from the collected optical data for the elapsed period of time; and calculating the physiological signal from the time series.

Optionally the optical data comprises video data, and wherein said obtaining said optical data comprises obtaining video data of the face of the subject. Optionally obtaining said optical data further comprises obtaining video data from a mobile phone camera, such that said camera comprises a mobile phone camera. Optionally said computational device comprises a mobile communication device. Optionally said mobile phone camera comprises a front facing camera. Optionally said computational device is physically separate from, but in communication with, said mobile phone camera. Optionally in combination with any method or a portion thereof as described herein, said detecting said optical data from said skin of the face comprises determining a plurality of face boundaries, selecting the face boundary with the highest probability and applying a histogram analysis to video data from the face.

Optionally said determining said plurality of face boundaries comprises applying a multi-parameter convolutional neural net (CNN) to said video data to determine said face boundaries. Optionally in combination with any method or a portion thereof as described herein, said obtaining said optical data further comprises obtaining video data of the skin of a finger of the subject. Optionally said obtaining said video data comprises obtaining video data of the skin of a fingertip of the subject by placing said fingertip on said camera. Optionally said camera for obtaining video data of said fingertip comprises a mobile phone camera. Optionally said mobile phone camera comprises a rear facing camera. Optionally said fingertip on said mobile phone camera further comprises activating a flash associated with said mobile phone camera to provide light.

Optionally in combination with any method or a portion thereof as described herein, said detecting said optical data from said skin of the face comprises determining a plurality of face or fingertip boundaries, selecting the face or fingertip boundary with the highest probability and applying a histogram analysis to video data from the face or fingertip. Optionally said determining said plurality of face or fingertip boundaries comprises applying a multi-parameter convolutional neural net (CNN) to said video data to determine said face or fingertip boundaries. Optionally the method may further comprise combining analyzed data from images of the face and fingertip to determine the physiological measurement.

Optionally in combination with any method as described herein, said determining the physiological signal further comprises combining meta data with measurements from said at least one physiological signal, wherein said meta data comprises one or more of weight, age, height, biological gender, body fat percentage and body muscle percentage of the subject. Optionally in combination with any method as described herein, said physiological signal is selected from the group consisting of stress, blood pressure, breath volume, and pSO2 (oxygen saturation).

According to at least some embodiments, there is provided a system for obtaining a physiological signal from a subject, the system comprising: a camera for obtaining optical data from a face of the subject, a user computational device for receiving optical data from said camera, wherein said user computational device comprises a processor and a memory for storing a plurality of instructions, wherein said processor executes said instructions for analyzing the optical data to select data related to the face of the subject, detecting optical data from a skin of the face, determining a time series from the optical data by collecting the optical data until an elapsed period of time has been reached and then calculating the time series from the collected optical data for the elapsed period of time; and calculating the physiological signal from the time series. Optionally said memory is configured for storing a defined native instruction set of codes and said processor is configured to perform a defined set of basic operations in response to receiving a corresponding basic instruction selected from the defined native instruction set of codes stored in said memory; wherein said memory stores a first set of machine codes selected from the native instruction set for analyzing the optical data to select data related to the face of the subject, a second set of machine codes selected from the native instruction set for detecting optical data from a skin of the face, a third set of machine codes selected from the native instruction set for determining a time series from the optical data by collecting the optical data until an elapsed period of time has been reached and then calculating the time series from the collected optical data for the elapsed period of time; and a fourth set of machine codes selected from the native instruction set for calculating the physiological signal from the time series.

Optionally said detecting said optical data from said skin of the face comprises determining a plurality of face boundaries, selecting the face boundary with the highest probability and applying a histogram analysis to video data from the face, such that said memory further comprises a fifth set of machine codes selected from the native instruction set for detecting said optical data from said skin of the face comprises determining a plurality of face boundaries, a sixth set of machine codes selected from the native instruction set for selecting the face boundary with the highest probability and a seventh set of machine codes selected from the native instruction set for applying a histogram analysis to video data from the face.

Optionally said determining said plurality of face boundaries comprises applying a multi-parameter convolutional neural net (CNN) to said video data to determine said face boundaries, such that said memory further comprises an eighth set of machine codes selected from the native instruction set for applying a multi-parameter convolutional neural net (CNN) to said video data to determine said face boundaries.

Optionally in combination with any system or a portion thereof as described herein, said camera comprises a mobile phone camera and wherein said optical data is obtained as video data from said mobile phone camera. Optionally said computational device comprises a mobile communication device. Optionally said mobile phone camera comprises a rear facing camera and a fingertip of the subject is placed on said camera for obtaining said video data. Optionally the system further comprises a flash associated with said mobile phone camera to provide light for obtaining said optical data.

Optionally said memory further comprises a ninth set of machine codes selected from the native instruction set for determining a plurality of face or fingertip boundaries, a tenth set of machine codes selected from the native instruction set for selecting the face or fingertip boundary with the highest probability, and an eleventh set of machine codes selected from the native instruction set for applying a histogram analysis to video data from the face or fingertip.

Optionally said memory further comprises a twelfth set of machine codes selected from the native instruction set for applying a multi-parameter convolutional neural net (CNN) to said video data to determine said face or fingertip boundaries. Optionally the system further comprises combining analyzed data from images of the face and fingertip to determine the physiological measurement according to said instructions executed by said processor. Optionally in combination with any system or a portion thereof as described herein, the system further comprises a display for displaying the physiological measurement and/or signal. Optionally said user computational device further comprises said display.

Optionally in combination with any system or a portion thereof as described herein, said user computational device further comprises a transmitter for transmitting said physiological measurement and/or signal. Optionally in combination with any system or a portion thereof as described herein, said determining the physiological signal further comprises combining meta data with measurements from said at least one physiological signal, wherein said meta data comprises one or more of weight, age, height, biological gender, body fat percentage and body muscle percentage of the subject. Optionally in combination with any system or a portion thereof as described herein, said physiological signal is selected from the group consisting of stress, blood pressure, breath volume, and pSO2 (oxygen saturation).

According to at least some embodiments, there is provided a system for obtaining a physiological signal from a subject, the system comprising: a rear facing camera for obtaining optical data from a finger of the subject, a user computational device for receiving optical data from said camera, wherein said user computational device comprises a processor and a memory for storing a plurality of instructions, wherein said processor executes said instructions for analyzing the optical data to select data related to the face of the subject, detecting optical data from a skin of the finger, determining a time series from the optical data by collecting the optical data until an elapsed period of time has been reached and then calculating the time series from the collected optical data for the elapsed period of time; and calculating the physiological signal from the time series. Optionally the system further comprises any system or portion thereof as described herein.

According to at least some embodiments, there is provided a method for obtaining a physiological signal from a subject, comprising operating any system as described herein, to obtain said physiological signal from said subject.

Implementation of the method and system of the present invention involves performing or completing certain selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

Although the present invention is described with regard to a “computing device”, a “computer”, or “mobile device”, it should be noted that optionally any device featuring a data processor and the ability to execute one or more instructions may be described as a computer, including but not limited to any type of personal computer (PC), a server, a distributed server, a virtual server, a cloud computing platform, a cellular telephone, an IP telephone, a smartphone, or a PDA (personal digital assistant). Any two or more of such devices in communication with each other may optionally comprise a “network” or a “computer network”.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the drawings:

FIGS. 1A and 1B show exemplary non-limiting illustrative systems for obtaining video data of a user and for analyzing the video data to determine one or more biological signals;

FIG. 2 shows a non-limiting exemplary method for performing signal analysis;

FIGS. 3A and 3B show non-limiting exemplary methods for enabling the user to use the app to obtain biological statistics;

FIG. 4 shows a non-limiting exemplary process for creating detailed biological statistics;

FIGS. 5A-5E show a non-limiting, exemplary method for obtaining video data and then performing the initial processing;

FIG. 6A relates to a non-limiting exemplary method for pulse rate estimation and determination of the rPPG, while FIGS. 6B-6C relate to some results of this method;

FIG. 7 shows a non-limiting exemplary method for performing an HRV or heart rate variability time domain analysis; and

FIG. 8 shows a non-limiting exemplary method for calculating the heart rate variability or HRV frequency domain.

### DESCRIPTION OF AT LEAST SOME EMBODIMENTS

A key underlying problem for rPPG mechanisms is accurate face detection and precise skin surface selection suitable for analysis, if images of the face are used. Similar problems are encountered if images of the fingertip are used, for example for images taken with the rear facing camera of a mobile device, such as a smart phone for example. The presently claimed invention overcomes this problem for face, finger and skin detection based on neural network methodology. Non-limiting examples are provided below. Preferably, for the skin selection, a histogram based algorithm used. Applying this procedure on part of the video frame containing the face only (or alternatively the finger only), the mean values for each channel, Red, Green, and Blue (RGB) construct the frame data. When using above procedures continuously for consequent video frames, the time series of RGB data is obtained. Each element of these time series represented by RGB values is obtained frame by frame, with time stamps used to determine elapsing time from the first occurrence of the first element. Then, the rPPG analysis begins when the total elapsed time reaches the averaging period used for the pulse rate estimation defined external parameter, for a complete a time window (Lalgo). Taking into account the variable frame acquisition rate, the time series data has to be interpolated with respect to the fixed given frame rate.

After interpolation, a pre-processing mechanism is applied to construct more suitable three dimensional signal (RGB). Such pre-processing may include for example normalization and filtering. Following pre-processing, the rPPG trace signal is calculated, including estimating the mean pulse rate.

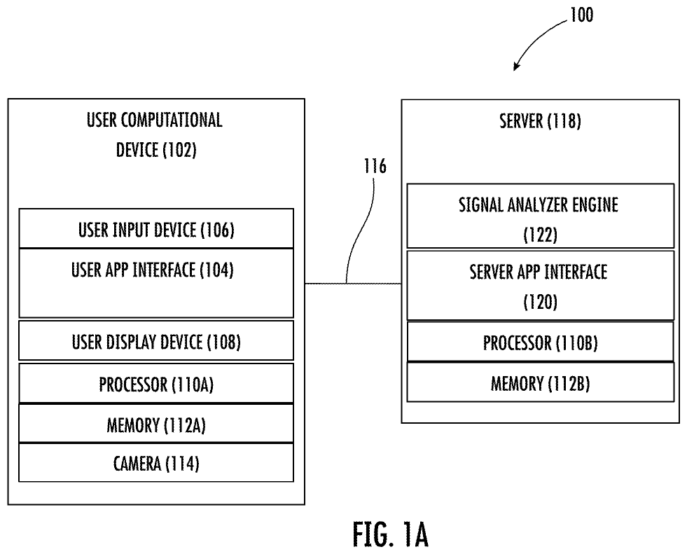

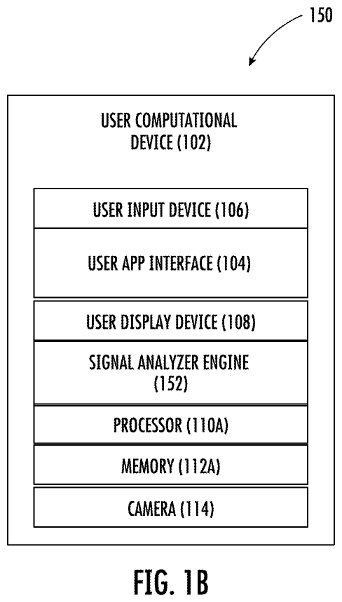

Turning now to the drawings, FIGS. 1A and 1B show exemplary non-limiting illustrative systems for obtaining video data of a user and for analyzing the video data to determine one or more biological signals.

FIG. 1A shows a system 100 featuring a user computational device 102, communicating with a server 118. The user computational device 102 preferably communicates with a server 118 through a computer network 116. User computational device 102 preferably includes user input device 106, which may include, for example, a pointing device such as a mouse, keyboard, and/or other input device.

In addition, user computational device 102 preferably includes a camera 114, for obtaining video data of a face of the user. The camera may also be separate from the user computational device. Optionally camera 114 comprises a rear facing camera of a mobile device, or another type of camera that is suitable for obtaining video data of a finger of the user, preferably of a portion of the finger such as a fingertip for example. User computational device 102 may comprise one or both of such cameras. The user interacts with a user app interface 104, for providing commands for determining the type of signal analysis, for starting the signal analysis, and for also receiving the results of the signal analysis.

For example, the user may, through user computational device 102, start recording video data through camera 114, either by separately activating camera 114, or by recording such data by issuing a command through user app interface 104.

Next, the video data is preferably sent to server 118, where it is received by server app interface 120. It is then analyzed by signal analyzer engine 122. Signal analyzer engine 122 preferably includes detection of the face in the video signals, followed by skin detection. Alternatively or additionally, signal analyzer engine 122 preferably includes detection of the finger or a portion thereof, such as the fingertip, in the video signals, followed by skin detection. As described in detail below, various non-limiting algorithms are preferably applied to support obtaining the pulse signals from this information. Next, the pulse signals are preferably analyzed according to time, frequency and non-linear filters to support the determination of HRV. Further analyses may then be performed from the HRV determination.

User computational device 102 preferably features a processor 110A, and a memory 112A. Server 118 preferably features a processor 110B, and a memory 112B.

As used herein, a processor such as processor 110A or 110B generally refers to a device or combination of devices having circuitry used for implementing the communication and/or logic functions of a particular system. For example, a processor may include a digital signal processor device, a microprocessor device, and various analog-to-digital converters, digital-to-analog converters, and other support circuits and/or combinations of the foregoing. Control and signal processing functions of the system are allocated between these processing devices according to their respective capabilities. The processor may further include functionality to operate one or more software programs based on computer-executable program code thereof, which may be stored in a memory, such as memory 112A or 112B in this non-limiting example. As the phrase is used herein, the processor may be “configured to” perform a certain function in a variety of ways, including, for example, by having one or more general-purpose circuits perform the function by executing particular computer-executable program code embodied in computer-readable medium, and/or by having one or more application-specific circuits perform the function.

Optionally, memory 112A or 112B is configured for storing a defined native instruction set of codes. Processor 110A or 110B is configured to perform a defined set of basic operations in response to receiving a corresponding basic instruction selected from the defined native instruction set of codes stored in memory 112A or 112B. Optionally memory 112A or 112B stores a first set of machine codes selected from the native instruction set for analyzing the optical data to select data related to the face of the subject, a second set of machine codes selected from the native instruction set for detecting optical data from a skin of the face, a third set of machine codes selected from the native instruction set for determining a time series from the optical data by collecting the optical data until an elapsed period of time has been reached and then calculating the time series from the collected optical data for the elapsed period of time; and a fourth set of machine codes selected from the native instruction set for calculating the physiological signal from the time series.

Optionally memory 112A or 112B further comprises a fifth set of machine codes selected from the native instruction set for detecting said optical data from said skin of the face comprises determining a plurality of face boundaries, a sixth set of machine codes selected from the native instruction set for selecting the face boundary with the highest probability and a seventh set of machine codes selected from the native instruction set for applying a histogram analysis to video data from the face.

Optionally memory 112A or 112B further comprises an eighth set of machine codes selected from the native instruction set for applying a multi-parameter convolutional neural net (CNN) to said video data to determine said face boundaries.

In addition, user computational device 102 may feature user display device 108 for displaying the results of the signal analysis, the results of one or more commands being issued and the like.

FIG. 1B shows a system 150, in which the above described functions are performed by user computational device 102. For either of FIG. 1A or 1B, user computational device 102 may comprise a mobile phone. In FIG. 1B, the previously described signal analyzer engine is now operated by user computational device 102 as signal analyzer engine 152. Signal analyzer engine 152 may have the same or similar functions to those described for signal analyzer engine in FIG. 1A. In FIG. 1B, user computational device 102 may be connected to a computer network such as the internet (not shown) and may also communicate with other computational devices. In at least some embodiments, some of the functions are performed by user computational device 102 while others are performed by a separate computational device, such as a server for example (not shown in FIG. 1B, see FIG. 1A).

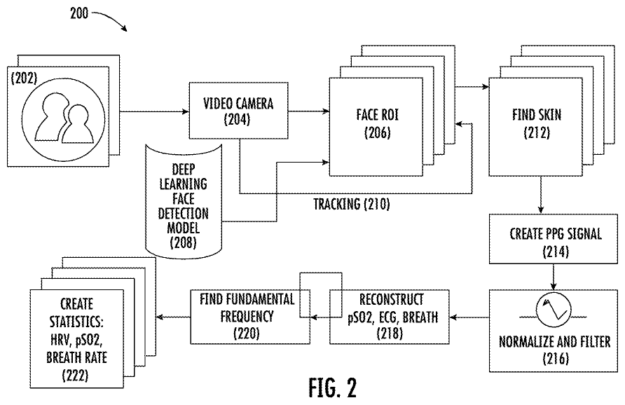

FIG. 2 shows a non-limiting exemplary method for performing signal analysis. A process 200 begins by initiating the process of obtaining data at block 202, for example, by activating a video camera 204. Face recognition is then optionally performed at 206, to first of all locate the face of the user. This may, for example, be performed through a deep learning face detection module 208, and also through a tracking process 210. It is important to locate the face of the user, as the video data is preferably of the face of the user in order to obtain the most accurate results for signal analysis. Tracking process 210 is based on a continuous features matching mechanism. The features represent a previously detected face in a new frame. The features are determined according to the position in the frame and from the output of an image recognition process, such as a CNN (convolutional neural network). When only one face appears in the frame, tracking process 210 can be simplified to face recognition within the frame.

As a non-limiting example, optionally, a Multi-task Convolutional Network algorithm is applied for face detection which achieves state-of-the-art accuracy under real-time conditions. It is based on the network cascade that was introduced in a publication by Li et al (Haoxiang Li, Zhe Lin, Xiaohui Shen, Jonathan Brandt, and Gang Hua. A convolutional neural network cascade for face detection. In The IEEE Conference on Computer Vision and Pattern Recognition (CVPR), June 2015).

Next, the skin of the face of the user is located within the video data at 212. Preferably, for the skin selection, a histogram based algorithm used. Applying this procedure on part of the video frame containing the face only, as determined according to the previously described face detection algorithm, the mean values for each channel, Red, Green, and Blue (RGB) are preferably used to construct the frame data.

Alternatively or additionally, the same or a similar process may be used for analysis of images of the finger or a portion thereof, such as the fingertip for example. For example, video data is obtained in a data block as described above, but of images of the finger or a portion thereof, such as the fingertip. The process of face recognition is then preferably adapted at 206, to first of all locate the finger or the portion thereof of the user. This process may, for example, be performed through an adapted finger or finger portion detection module (not shown), and also through an adapted tracking process (not shown) which is adapted to track the finger or the portion thereof through different images. Optionally, if the fingertip is pressed directly against the rear camera of a mobile device, tracking may be less necessary, although preferably fingertip recognition is still performed. In any case, preferably the skin of the fingertip is located as described for example for the above process for face recognition. Also preferably, if images of the finger or a portion thereof are to be analyzed, preferably the skin thereof is located as described for example for the above process for face recognition.

When using above procedures continuously for consequent video frames, a time series of RGB data is obtained. Each frame, with its RGB values, represents an element of these time series. Each element has a time stamp determined according to elapsed time from the first occurrence. The collected elements may be described as being in a scaled buffer having L algo elements. The frames are preferably collected until sufficient elements are collected. The sufficiency of the number of elements is preferably determined according to the total elapsed time. The rPPG analysis of 214 begins when the total elapsed time reaches the length of time required for the averaging period used for the pulse rate estimation. The collected data elements may be interpolated. Following interpolation, the pre-processing mechanism is preferably applied to construct a more suitable three dimensional signal (RGB).

A PPG signal is created at 214 from the three dimensional signal and specifically from the elements of the RGB data. For example, the pulse rate may be determined from a single calculation or from a plurality of cross-correlated calculations, as described in greater detail below. This may be then normalized and filtered at 216, and may be used to reconstruct PSO₂, ECG, and breath at 218. A fundamental frequency is found at 220, and the statistics are created such as heart rate, PSO₂, and breath rates and so forth at 222.

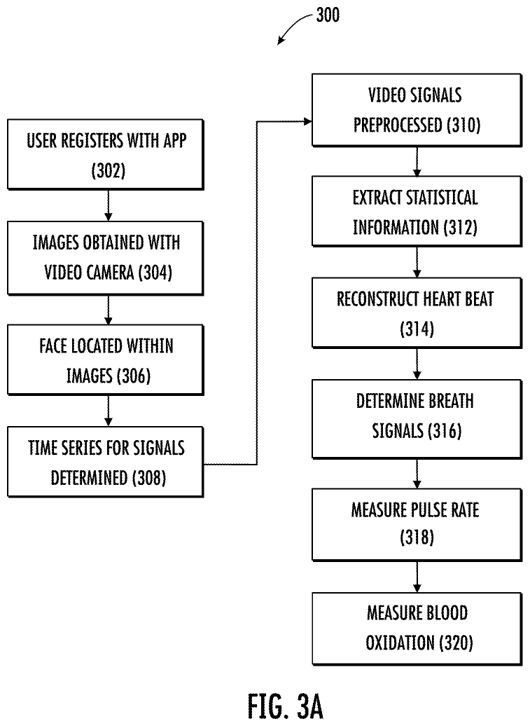

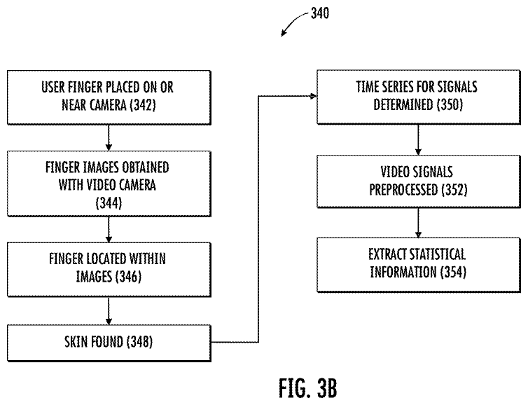

FIGS. 3A and 3B show non-limiting exemplary methods for enabling the user to use the app to obtain biological statistics. FIG. 3A shows a non-limiting exemplary method using optical images of the face alone. FIG. 3B shows a similar, non-limiting, exemplary method for analyzing video data of the fingertip of the user, for example from the rear camera of a mobile device as previously described. Optionally both methods may be combined.

Turning now to the drawings, as shown in FIG. 3A, in a method 300, the user registers with the app at 302. Next, images are obtained with the video camera, for example as attached to or formed with user computational device at 304. The video camera is preferably a RGB camera as described herein.

The face is located within the images 306. This may be performed on the user computational device, at a server, or optionally at both. Furthermore, this process may be performed as previously described, with regard to a multi-task convolutional neural net. Skin detection is then performed, by applying a histogram to the RGB signal data. Only the video data relating to light reflected from the skin is preferably analyzed for optical pulse detection and HRV determination.

The time series for the signals are determined at 308, for example as previously described. Taking into account the variable frame acquisition rate, the time series data is preferably interpolated with respect to the fixed given frame rate. Before running the interpolation procedure, preferably the following conditions are analyzed so that interpolation can be performed. First, preferably the number of frames is analyzed to verify that after interpolation and pre-processing, there will be enough frames for the rPPG analysis.

Next, the frames per second are considered, to verify that the measured frames per second in the window is above a minimum threshold. After that, the time gap between frames, if any, is analyzed to ensure that it is less than some externally set threshold, which for example may be 0.5 seconds.

If any of the above conditions not satisfied, then the procedure preferably terminates with full data reset and restarts from the last valid frame, for example to return to 304 as described above.

Next the video signals are preferably pre-processed at 310, following interpolation. The pre-processing mechanism is applied to construct a more suitable three dimensional signal (RGB). The pre-processing preferably includes normalizing each channel to the total power; scaling the channel value by its mean value (estimated by low pass filter) and subtracting by one; and then passing the data through a Butterworth band pass IIR filter.

Statistical information is extracted at 312. A heartbeat is then reconstructed at 314. Breath signals are determined at 316, and then the pulse rate is measured at 318. After this, the blood oxidation is measured at 320.

FIG. 3B shows a similar, non-limiting, exemplary method for analyzing video data of the fingertip of the user, for example from the rear camera of a mobile device as previously described. This process may be used for example if sufficient video data cannot be captured from the front facing camera, for the face of the user. Optionally both methods may be combined.

In a method 340, the method begins by placing the fingertip of the user on or near the camera at 342. If near the camera, then the fingertip needs to be visible to the camera. This placement may be accomplished for example in a mobile device, by having the user place the fingertip on the rear camera of the mobile device. The camera is already in a known geometric position in relation to placement of the fingertip, which encourages correct placement of the fingertip in terms of collecting accurate video data. Optionally the flash of the mobile device may be enabled in a longer mode (“torch” or “flashlight” mode) to provide sufficient light. Enabling the flash may be performed automatically if sufficient light is not detected by the camera for accurate video data of the fingertip to be obtained.

At 344, images of the finger, and preferably of the fingertip, are obtained with the camera. Next the finger, and preferably the fingertip, is located within the images at 346. This process may be performed as previously described with regard to location of the face within the images. However, if a neural net is used, it will need to be trained specifically to locate fingers and preferably fingertips. Hand tracking from optical data is known in the art; a modified hand tracking algorithm could be used to track fingertips within a series of images.

At 348, the skin is found within the finger, and preferably fingertip, portion of the image. Again, this process may be performed generally as described above for skin location, optionally with adjustments for finger or fingertip skin. The time series for the signals are determined at 350, for example as previously described but preferably adjusted for any characteristics of using the rear camera and/or the direct contact of the fingertip skin on the camera. Taking into account the variable frame acquisition rate, the time series data is preferably interpolated with respect to the fixed given frame rate. Before running the interpolation procedure, preferably the following conditions are analyzed so that interpolation can be performed. First, preferably the number of frames is analyzed to verify that after interpolation and pre-processing, there will be enough frames for the rPPG analysis.

Next, the frames per second are considered, to verify that the measured frames per second in the window is above a minimum threshold. After that, the time gap between frames, if any, is analyzed to ensure that it is less than some externally set threshold, which for example may be 0.5 seconds.

If any of the above conditions is not satisfied, then the procedure preferably terminates with full data reset and restarts from the last valid frame, for example to return to 344 as described above.

Next the video signals are preferably pre-processed at 352, following interpolation. The pre-processing mechanism is applied to construct a more suitable three dimensional signal (RGB). The pre-processing preferably includes normalizing each channel to the total power; scaling the channel value by its mean value (estimated by low pass filter) and subtracting by one; and then passing the data through a Butterworth band pass IIR filter. Again, this process is preferably adjusted for the fingertip data. At 354, statistical information is extracted, after which the process may proceed for example as described with regard to FIG. 3A above, from 314, to reconstruct the heart beat and to perform other measurements as described herein.

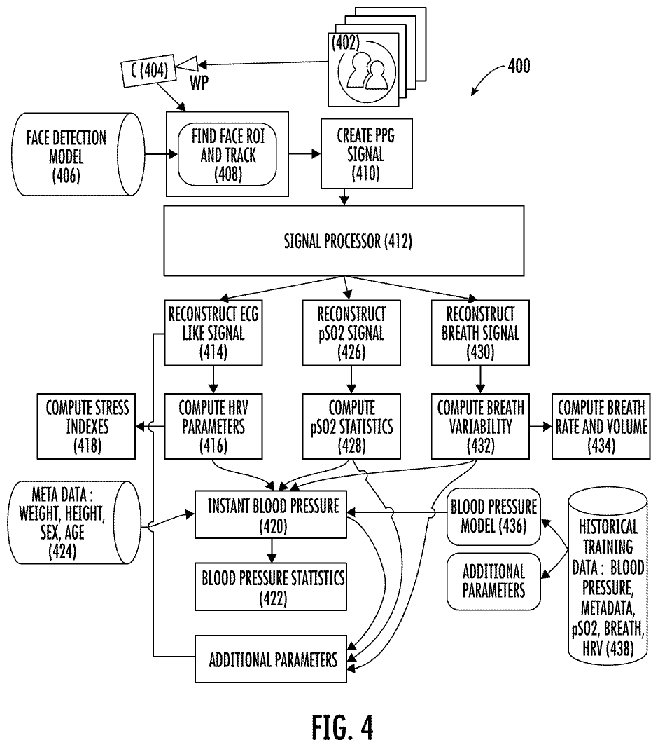

FIG. 4 shows a non-limiting exemplary process for creating detailed biological statistics. In a process 400, user video data is obtained through a user computational device 402, with a camera 404. A face detection model 406 is then used to find the face. For example, after face video data has been detected for a plurality of different face boundaries, all but the highest-scoring face boundary is preferably discarded. Its bounding box is cropped out of the input image, such that data related to the user's face is preferably separated from other video data. Skin pixels are preferably collected using a histogram based classifier with a soft thresholding mechanism, as previously described. From the remaining pixels, the mean value is computed per channel, and then passed on to the rPPG algorithm at 410. This process enables skin color to be determined, such that the effect of the pulse on the optical data can be separated from the effect of the underlying skin color. The process tracks the face at 408 according to the highest scoring face bounding box.

As noted above, this process may be adapted to detect the finger or portion thereof, such as the fingertip for example. Preferably a boundary detecting algorithm is also used to detect the boundaries of the finger or portion thereof, such as the fingertip. The subsequent processes, such as cropping out the bounding box to separate the relevant portion of the user's anatomy, such as the finger or portion thereof, such as the fingertip for example. An adapted histogram based classifier may also be used, given that the relevant portions of the anatomy being detected, such as the fingertip for example, comprise skin. The process at 408 may be adapted if the user presses a fingertip against the rear camera, for example to accommodate a reduced need for tracking, given the direct placement of the fingertip against the rear camera.

Next, the PPG signals are created at 410. Following pre-processing, the rPPG trace signal is calculated using a L algo elements of the scaled buffer. The procedure is described as follows: The mean pulse rate is estimated using a match filter between two rPPG different analytic signals constructed from raw interpolated data (CHROM like and Projection Matrix (PM)). Then the cross-correlation is calculated on which the mean instantaneous pulse rate is searched. Frequency estimation is based on non-linear least square (NLS) spectral decomposition with additional lock-in mechanism. The rPPG signal, then is derived from the PM method applying adaptive Wiener filtering and with initial guess signal to be the dependent on instantaneous pulse rate frequency (vpr): sin(2πvprn). Further, an additional filter in the frequency domain used to force signal reconstruction. Lastly, the exponential filter applied on instantaneous RR values obtained by procedure discussed in greater detail below.

The signal processor at 412 then preferably performs a number of different functions, based on the PPG signals. These preferably include reconstructing an ECG-like signal at 414, computing the HRV (heart rate variability) parameters at 416, and then computing a stress index at 418.

HRV is the physiological phenomenon of variation in the time interval between heartbeats. It is measured by the variation in the beat-to-beat interval. Other terms used include: “cycle length variability”, “RR (NN) variability” (where R is a point corresponding to the peak of the QRS complex of the ECG wave; and RR is the interval between successive Rs), and “heart period variability”.

As described in greater detail below, it is possible to calculate 24 h, semi(˜15 min), short-term (ST, ˜5 min) or brief, and ultra-short-term (UST, <5 min) HRV using time-domain, frequency-domain, and non-linear measurements.

In addition, the instant blood pressure may be created at 420, followed by blood pressure statistics 422. Optionally metadata at 424 is included in this calculation. The metadata may for example relate to height, weight, gender or other physiological or demographic data. At 426, the PS0₂ signal is reconstructed, followed by computing the PS0₂ statistics at 428. The statistics at 428 may then lead to the further blood pressure analysis as previously described with regard to 420 and 422.

Optionally a breath signal is reconstructed at 430 by the previously described signal processor 412, followed by computing the breath variability at 432. The breath rate and volume are then preferably calculated at 434.

From the instant blood pressure calculations at 420, optionally a blood pressure model is calculated at 436. The calculation of the blood pressure model may be influenced or adjusted according to historical data at 438, such as previously determined blood pressure, breath rate and volume, PS0₂, or other calculations.

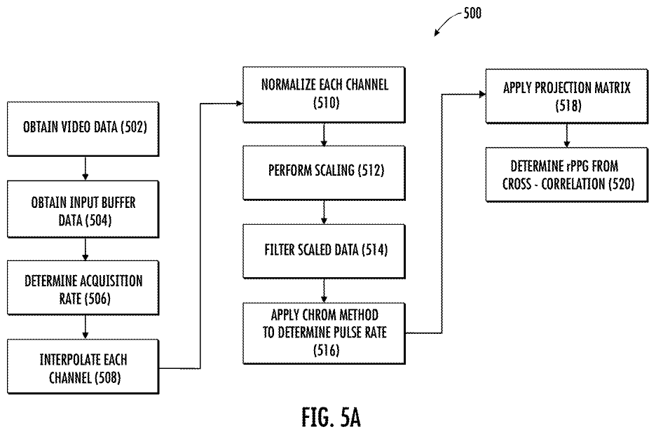

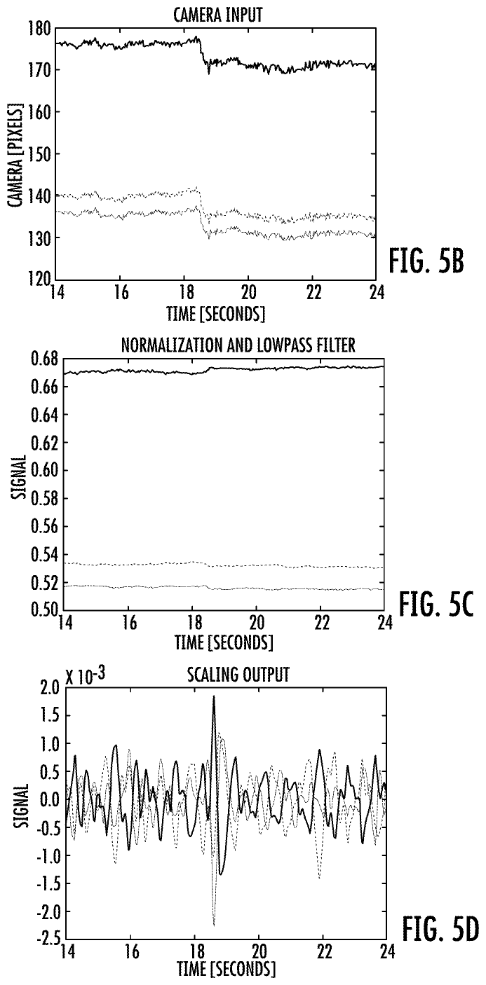

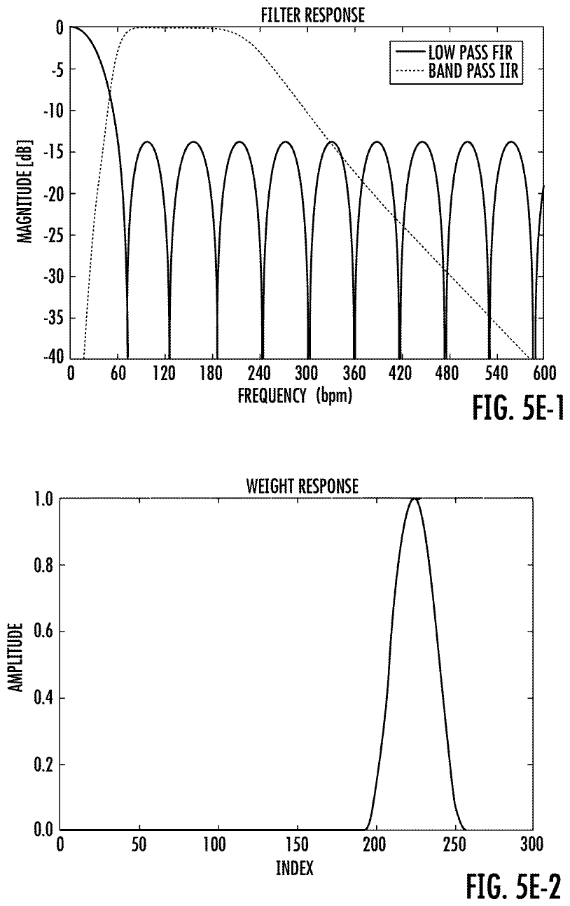

FIGS. 5A-5E show a non-limiting, exemplary method for obtaining video data and then performing the initial processing, which preferably includes interpolation, pre-processing and rPPG signal determination, with some results from such initial processing. Turning now to FIG. 5A, in a process 500, video data is obtained in 502, for example as previously described.

Next the camera channels input buffer data is obtained at 504, for example as previously described. Next a constant and predefined acquisition rate is preferably determined at 506. For example, the constant and predefined acquisition rate may be set at Δt=1/fps˜=33 ms. At 508, each channel is preferably interpolated separately to the time buffer with the constant and predefined acquisition rate. This step removes the input time jitter. Even though the interpolation procedure adds aliasing (and/or frequency folding), aliasing (and/or frequency folding) has already occurred once the images were taken by the camera. The importance of interpolating into a constant sample rate is that it satisfies a basic assumption of quasi-stationarity of the heart rate in accordance to the acquisition time. The method used for interpolation may for example be based on cubic Hermite interpolation.

FIGS. 5B-5D show data relating to different stages of the scaling procedure. The color coding corresponds to the colors of each channel, i.e. red corresponds to the red channel (top line; middle line is green; lower line is blue) and so forth. FIG. 5B shows the camera channel data after interpolation.

Turning back to FIG. 5A, at 510-514, after interpolating each of the colored channels (vec(c)), pre-processing is performed to enhance the pulsatile modulations. The pre-processing preferably incorporates three steps. At 510, normalization of each channel to the total power is performed, which reduces noise due to overall external light modulation.

The power normalization is given by

$${\vec{c}}_{p} = \frac{\vec{c}}{\sqrt{{c}_{r}^{2} + {c}_{g}^{2} + {c}_{b}^{2}}} \qquad ( 1 )$$

with—→c p is the power normalized camera channel vector, and—→c is the interpolated input vector as described. For brevity reason, the frame index was removed from both sides.

Next, at 512, scaling is performed. For example, such scaling may be performed by the mean value i and subtracted by one, which reduces effects of stationary light source and its brightness level. The mean value is set by the segment length (Lalgo), but this type of a solution can enhance low frequency components. Alternatively, instead of scaling by the mean value, it is possible to scale by a low pass FIR filter.

Using a low pass filter adds an inherent latency, which requires compensation on M/2 frames. The scaled signal is given by:

$${c}_{s} ( n - \frac{M}{2} ) = \frac{{c}_{p} ( n - \frac{M}{2} )}{{\sum }_{m = 0}^{m = M} b ( m ) {c}_{p} ( n - m )} - 1 \qquad ( 2 )$$

with cs(n) is a single channel scaled value of frame n, and b is the lowpass FIR coefficients. The channel color notation was removed from the above formula for brevity.

At 514, the scaled data is passed through Butterworth band pass IIR filter.

This filter is defined as:

$$s ( n ) = \text{?} b ( m ) {c}_{s} ( n - m ) - {\sum }_{l = 1}^{l = {M}_{fb}} a ( l ) s ( n - l ) \qquad ( 3 )$$

$$\text{?} \text{indicates text missing or illegible when filed}$$

The output of the scaling procedure is—→s each new frame adds a new frame with latency for each camera channel. Note that for brevity the frame index n is used but it actually refers to frame n—M/2 (due to the low pass filter).

FIG. 5C shows power normalization of the camera input, plot of the low-pass scaled data before the band-pass filter (top line is red; middle line is green; lower line is blue). FIG. 5D shows a plot of the power scaled data before the band-pass filter. FIG. 5E shows a comparison of the mean absolute deviation for all subjects using the two normalization procedures, with the filter response given as FIG. 5E-1 and the weight response (averaging by the mean) given as FIG. 5E-2. FIG. 5E-1 shows the magnitude and frequency response of the pre-processing filters. The blue line represents the M=33 tap low pass FIR filter (line starting at the left most side), while the red line shows the third order IIR Butterworth filter. FIG. 5E-2 shows the 64 long Hann window weight response used for averaging the rPPG trace.

At 516 the CHROM algorithm is applied to determine the pulse rate. This algorithm is applied by projecting the signals onto two planes defined by

$${S}_{c , 1} = 3 {s}_{r} - 2 {s}_{q} \qquad ( 4 )$$

$${S}_{c , 2} = 1.5 {s}_{r} + {s}_{g} - 1.5 {s}_{b} \qquad ( 5 )$$

$$( 6 )$$

Then the rPPG signal is taken as the difference between the two

$$chrom = {S}_{c , 1} - \frac{σ ( {S}_{c , 1} )}{σ ( {S}_{c , 2} )} {S}_{c , 2} \qquad ( 7 )$$

with σ( . . . ) is the standard deviation of the signal. Note that the two projected signals were normalized by their maximum fluctuation. The CHROM method is derived to minimize the specular light reflection.

Next at 518 the projection matrix is applied to determine the pulse rate. For the projection matrix (PM) method the signal is projected to the pulsatile direction. Even though the three elements are not orthogonal, it was surprisingly found that this projection gives a very stable solution with better signal to noise than CHROM. To derive the PM method, the matrix elements of the intensity, specular, and pulsatile elements of the RGB signal are determined:

$${M}_{measured} = [ \begin{matrix}1 & 0.77 & 0.33 \\ 1 & 0.51 & 0.77 \\ 1 & 0.38 & 0.53\end{matrix} ] \qquad ( 8 )$$

The above matrix elements may be determined for example from a paper by de Haan and van Leest (G de Haan and A van Leest. Improved motion robustness of remote-ppg by using the blood volume pulse signature. Physiological Measurement, 35(9):1913, 2014). In this paper, the signals from arterial blood (and hence from the pulse) are determined from the RGB signals, and can be used to determine the blood volume spectra.

For this example the intensity is normalized to one. The projection to the pulsatile direction is found by inverting the above matrix and choosing the vector corresponding to the pulsatile. This gives:

pm=−0.26sᵣ−0.83s_{g}−0.50s_{b}

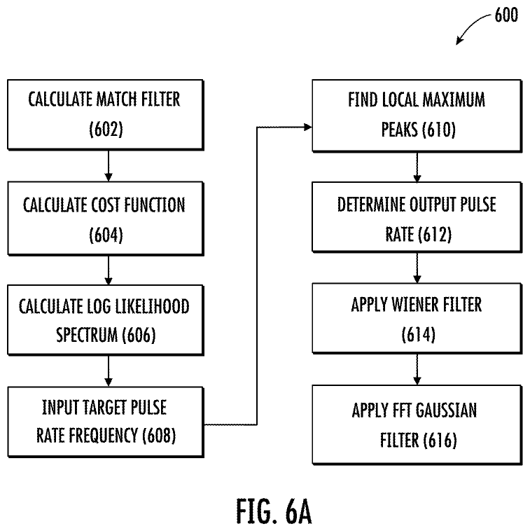

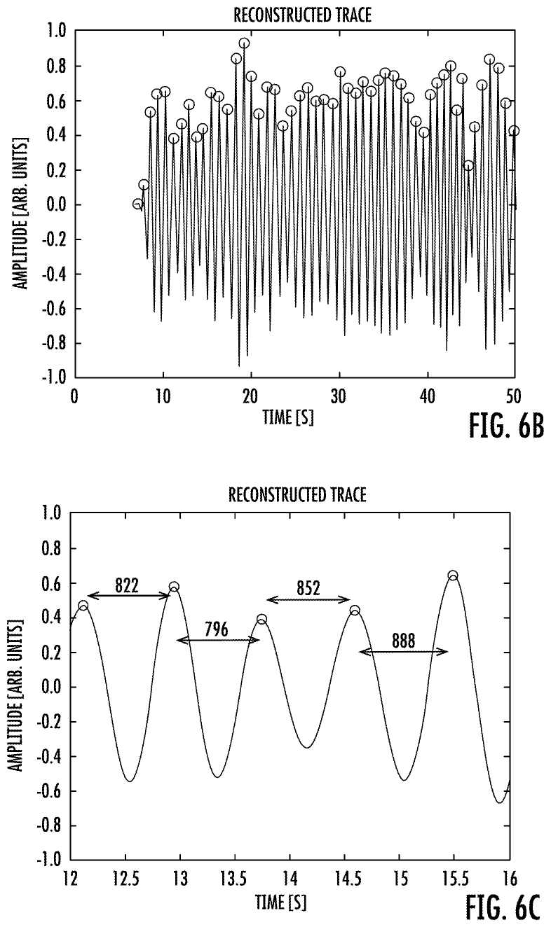

At 520, the two pulse rate results are cross-correlated to determine the rPPG. The determination of the rPPG is explained in greater detail with regard to FIG. 6.

FIG. 6A relates to a non-limiting exemplary method for pulse rate estimation and determination of the rPPG, while FIGS. 6B-6C relate to some results of this method. The method uses the output of the CHROM and PM rPPG methods, described above with regard to FIG. 5A, to find the pulse rate frequency vpr. This method involves searching for the mean pulse rate over the past Lalgo frames. The frequency is extracted from the output of a match filter (between the CHROM and PM), by using non-linear least square spectral decomposition with the application of a lock-in mechanism.

Turning now to FIG. 6A, in a method 600, the process begins at 602 by calculating the match filter between the CHROM and PM output. The match filter is simply done by calculating the correlation between CHROM and PM methods output. Next at 604, the cost function of a non-linear least squares (NLS) frequency estimation is calculated, based on a periodic function with its harmonics. In the above equation, x is the model output, al and bl are the weight of the frequency components, 1 is its harmonic order, L is number of orders in the model, v is the frequency, and (n) is the additive noise component. Then the log likelihood spectrum is calculated at 606 by adapting the algorithm given in Nielsen et. al (Jesper Kjær Nielsen, Tobias Lindstrom Jensen, Jesper Rindom Jensen, Mads Græesbøll Christensen, and Soren Holdt Jensen.

$$x ( n ) = \text{?} [ \text{?} cos ( 2 π l ν n ) - \text{?} sin ( 2 π l ν n ) ] + ϵ ( n ) \qquad ( 10 )$$

$$\text{?} \text{indicates text missing or illegible when filed}$$

Fast fundamental frequency estimation: Making a statistically efficient estimator computationally efficient. Signal Processing, 135:188-197, 2017) in a computational complexity of O(N log N)+O(NL).

In Nielsen et. A, the frequency is set as the frequency of the maximum peak out of all harmonic orders. The method itself is a general method, which can be adapted in this case by altering the band frequency parameters. An inherent feature of the model is that higher order will have more local maximum peaks in the cost function spectra than lower order. This feature is used for the lock-in procedure.

At 608, the lock-in mechanism gets as input the target pulse rate frequency vtraget. Then at 610, the method finds all the local maximum peaks amplitude (Ap) and frequency (vp) of the cost function spectrum of order 1=L. For each local maximum, the following function is estimated:

$$f ( {A}_{p} , {ν}_{p} , {ν}_{traget} ) = \frac{{A}_{p}}{\vert {ν}_{p} - {ν}_{traget} \vert} \qquad ( 11 )$$

This function takes a balance between the signal strength and distance from the target frequency. At 610, the output pulse rate is set as local peak vp which maximize the above function f (Ap,vp,vtraget)

FIGS. 6B and 6C show an exemplary reconstructed rPPG trace (blue line), of an example run. The red circles show the peak R time. FIG. 6B shows the trace from run start at time t=0s till time t=50s. FIG. 6C shows a zoom of the trace and showing also RR interval times in milliseconds.

Next at 612-614, the instantaneous rPPG signal is filtered, with two dynamic filters around the mean pulse rate frequency (vpr): Wiener filter, and FFT Gaussian filter. At 612, the

$$g ( ν ) = \text{?} \qquad ( 12 )$$

$$\text{?} \text{indicates text missing or illegible when filed}$$

Wiener filter is applied. The desired target is sin(2πvprn), with n is the index number (representing the time). At 614, the FFT Gaussian filter aims to clean the signal around vpr, thus a Gaussian shape of the form
is used with σg as its width. As the name suggests, the filtering is done by transforming the signal to its frequency domain (FFT) and multiplying it by g (v) and transforming back to the time domain and taking the real part component.

The output of the above procedure is a filtered rPPG trace (pm) of length Lalgo with mean pulse rate of vpr. The output is obtained for each observed video frame and constructing the overlapping time series of pulse. These time series must be averaged to produce mean final rPPG trace suitable for HRV processing. This is done using overlapping and addition of filtered rPPG signal (pm) using following formula (n represents time) from a paper by Wang et al (W. Wang, A. C. den Brinker, S. Stuijk, and G. de Haan. Algorithmic principles of remote ppg. IEEE Transactions on Biomedical Engineering, 64(7):1479-1491, July 2017):

t(n−Lalgo+1)=t(n−Lalgo+1)+w(1)pm(1) (13)

with 1 is a running index between 0 and Lalgo; where w(i) is a weight function, that sets the configuration and latency of the output trace. Obtaining then consequent peaks (maxima that represents systolic peak) it is possible construct so called RR intervals as distance in time. Using series of RR intervals is possible to retrieve HRV parameters as statistical measurements in both time and frequency domains.

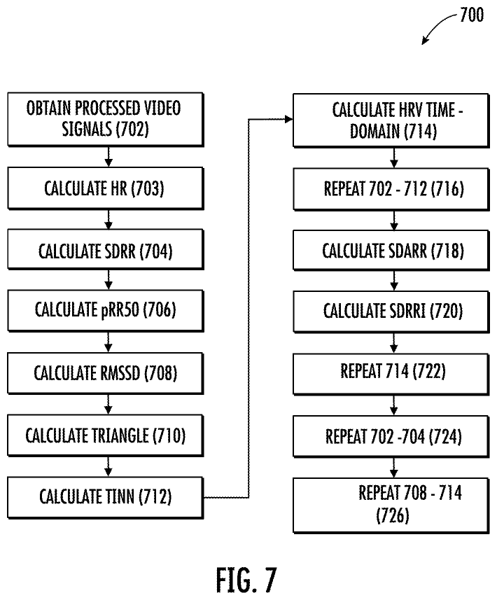

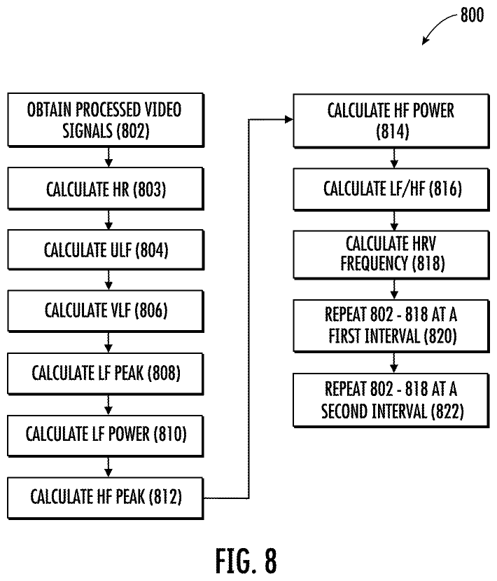

FIGS. 7 and 8 relate to methods for creating statistical measures for various parameters, which can then be used for providing the above information, such as for example calculating respiratory rate (RR). The tables relate to the standard set of HRV parameters and are calculated directly from RR intervals aggregated for different time periods. Most of these parameters refer to the statistical presentation of the HR variation in time.

FIG. 7 shows a non-limiting exemplary method for performing an HRV or heart rate variability time domain analysis. As shown in a method 700, processed video signals are obtained at 702. The processed video signals are then calculated to determine a heart rate (HR) at 703.

The SDRR is calculated at 704. The PRR50 is calculated at 706. The RMSSD is calculated at 708. The triangle is calculated at 710. The TINN is calculated at 712. The HRV heart rate variability time domain is calculated 714.

Steps 702-712 are preferred repeated at 716. The SDARR is calculated at 718. The SDRRI is calculated at 720. Steps 714-720 is optionally repeated at 722. Then steps 702-704 are optionally repeated at 724. Finally, steps 708-714 are optionally repeated at 726.

The meaning of the acronyms for the HRV time-domain measures are described below:

| | | | Calculated after X |
|---|---|---|---|
| Parameter | Unit | Description | minutes? |
| SDRR | Ms | Standard deviation of RR intervals | 2, 5, 15 min (each with |
| | | | a different meaning) |
| SDARR | Ms | Standard deviation of the average RR | 5 min |
| | | intervals for each 5 min segment of a 24 h | |
| | | HRV recording | |
| SDRR index | Ms | Mean of the standard deviations of all the | 5 min |
| (SDRRI) | | RR intervals for each 5 min segment of a | |
| | | 24 h HRV recording | |
| pRR50 | % | Percentage of successive RR intervals that | 2, 5 min (each with a |
| | | differ by more than 50 ms | different meaning) |
| RMSSD | Ms | Root mean square of successive RR | 2, 5, 15 min (each with |
| | | interval differences | a different meaning) |
| HRV triangular | | Integral of the density of the RR interval | 2, 5, 15 min (each with |
| index | | histogram divided by its height | a different meaning) |
| TINN | Ms | Baseline width of the RR interval | 2, 5, 15 min (each with |
| | | histogram | a different meaning) |

*Inter-beat interval, time interval between successive heartbeats; NN intervals, inter-beat intervals from which artifacts have been removed; RR intervals, inter-beat intervals between all successive heartbeats.

The following parameters may be calculated according to information provided in F. Shaffer and J. P. Ginsberg (An Overview of Heart Rate Variability Metrics and Norms, Front Public Health. 2017; 5: 258), which is hereby incorporated by reference as if fully set forth herein: SDRR, RMSSD, triangle (HRV triangular index), and TINN.

The following parameter may be calculated according to information provided in Umetani et al (Twenty-four hour time domain heart rate variability and heart rate: relations to age and gender over nine decades, J Am Coll Cardiol. 1998 Mar. 1;31(3):593-601): HRV time domain.

The following parameters may be calculated according to information provided in O. Murray (The Correlation Between Heart Rate Variability and Diet, Proceedings of The National Conference On Undergraduate Research (NCUR) 2016, North Carolina): SDRRI (SDRR index). SDARR and pRR50.

FIG. 8 shows a non-limiting exemplary method for calculating the heart rate variability or HRV frequency domain. In a method 800, processed video signals are obtained as previously described at 802. Heart rate is calculated as previously described at 803. The ULF is calculated at 804. The VLF is calculated at 806. The LF peak is calculated at 808.

LF power is calculated at 810. The HF peak is calculated at 812. HF power is calculated at 814. The ratio of LF to HF is calculated at 816. The HRV or heart rate variability frequency is calculated at 814. Steps 802-818 are optionally repeated at a first interval at 820. Then, steps 802-808 are optionally repeated at a second interval at 822.

The meaning of the acronyms for the HRV frequency-domain measures are described in greater detail below:

| | | | Calculated after X |
|---|---|---|---|
| Parameter | Unit | Description | minutes? |
| ULF power | ms2 | Absolute power of the ultra-low-frequency band | 2, 5, 15 min (each with |
| | | (≤0.003 Hz) | a different meaning) |
| VLF power | ms2 | Absolute power of the very-low-frequency band | 2, 5, 15 min (each with |
| | | (0.0033-0.04 Hz) | a different meaning) |
| LF peak | Hz | Peak frequency of the low-frequency band | 2, 5, 15 min (each with |
| | | (0.04-0.15 Hz) | a different meaning) |
| LF power | ms2 | Absolute power of the low-frequency band | 2, 5, 15 min (each with |
| | | (0.04-0.15 Hz) | a different meaning) |
| LF power | nu | Relative power of the low-frequency band | 2, 5, 15 min (each with |
| | | (0.04-0.15 Hz) in normal units | a different meaning) |
| LF power | % | Relative power of the low-frequency band | 2, 5, 15 min (each with |
| | | (0.04-0.15 Hz) | a different meaning) |
| HF peak | Hz | Peak frequency of the high-frequency band | 2, 5, 15 min (each with |
| | | (0.15-0.4 Hz) | a different meaning) |
| HF power | ms2 | Absolute power of the high-frequency band | 2, 5, 15 min (each with |
| | | (0.15-0.4 Hz) | a different meaning) |
| HF power | nu | Relative power of the high-frequency band | 2, 5, 15 min (each with |
| | | (0.15-0.4 Hz) in normal units | a different meaning) |
| HF power | % | Relative power of the high-frequency band | 2, 5, 15 min (each with |
| | | (0.15-0.4 Hz) | a different meaning) |
| LF/HF | % | Ratio of LF-to-HF power | 2, 5, 15 min (each with |
| | | | a different meaning) |

Additionally or alternatively, various non-linear measures may be determined for calculating HRV:

| | | | Calculated after X |
|---|---|---|---|
| Parameter | Unit | Description | minutes? |
| S | ms | Area of the ellipse which represents total HRV | over 5 min |
| SD1 | ms | Poincaré plot standard deviation perpendicular | over 5 min |
| | | the line of identity | |
| SD2 | ms | Poincaré plot standard deviation along the line of | over 5 min |
| | | identity | |
| SD1/SD2 | % | Ratio of SD1-to-SD2 | over 5 min |
| ApEn | | Approximate entropy, which measures the | over 5 min |
| | | regularity and complexity of a time series | |
| SampEn | | Sample entropy, which measures the regularity | over 5 min |
| | | and complexity of a time series | |
| DFA α1 | | Detrended fluctuation analysis, which describes | over 5 min |
| | | short-term fluctuations | |
| DFA α2 | | Detrended fluctuation analysis, which describes | over 5 min |
| | | long-term fluctuations | |
| D2 | | Correlation dimension, which estimates the | over 5 min |
| | | minimum number of variables required to | |
| | | construct a model of system dynamics | |

The following parameters may be calculated according to information provided in the previously described paper by F. Shaffer and J. P. Ginsberg: ULF, VLF, LF peak, LF power, HF peak, HF power, LF/HF and HRV frequency.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1-37. (canceled)

38. A method for obtaining a physiological signal from a subject, the method comprising obtaining optical data from a face of the subject with a camera, analyzing the optical data to select data related to the face of the subject with a computational device in communication with said camera, detecting optical data from a skin of the face, determining a time series from the optical data by collecting the optical data until an elapsed period of time has been reached and then calculating the time series from the collected optical data for the elapsed period of time; and calculating the physiological signal from the time series; wherein said detecting said optical data from said skin of the face comprises determining a plurality of face boundaries, selecting the face boundary with the highest probability and applying a histogram analysis to video data from the face; wherein said histogram analysis comprises a histogram based classifier with a soft thresholding mechanism.

39. The method of claim 38, wherein the optical data comprises video data, and wherein said obtaining said optical data comprises obtaining video data of the face of the subject.

40. The method of claim 39, wherein said obtaining said optical data further comprises obtaining video data from a mobile phone camera, such that said camera comprises a mobile phone camera.

41. The method of claim 40, wherein said computational device comprises a mobile communication device.

42. The method of claim 41, wherein said mobile phone camera comprises a front facing camera.

43. The method of claim 42, wherein said computational device is physically separate from, but in communication with, said mobile phone camera.

44. The method of claim 38, wherein said determining said plurality of face boundaries comprises applying a multi-parameter convolutional neural net (CNN) to said video data to determine said face boundaries.

45. The method of claim 38 wherein said obtaining said optical data further comprises obtaining video data of the skin of a finger of the subject.

46. The method of claim 45, wherein said obtaining said video data comprises obtaining video data of the skin of a fingertip of the subject by placing said fingertip on said camera.

47. The method of claim 46, wherein said camera for obtaining video data of said fingertip comprises a mobile phone camera.

48. The method of claim 47 wherein said fingertip on said mobile phone camera further comprises activating a flash associated with said mobile phone camera to provide light.

49. The method of claim 38, wherein said detecting said optical data from said skin of the face comprises determining a plurality of face or fingertip boundaries, selecting the face or fingertip boundary with the highest probability and applying a histogram analysis to video data from the face or fingertip.

50. The method of claim 49, wherein said determining said plurality of face or fingertip boundaries comprises applying a multi-parameter convolutional neural net (CNN) to said video data to determine said face or fingertip boundaries.

51. The method of claim 50, further comprising combining analyzed data from images of the face and fingertip to determine the physiological measurement.

52. The method of claim 51, wherein said determining the physiological signal further comprises combining meta data with measurements from said at least one physiological signal, wherein said meta data comprises one or more of weight, age, height, biological gender, body fat percentage and body muscle percentage of the subject.

53. The method of claim 52, wherein said physiological signal is selected from the group consisting of stress, blood pressure, breath volume, and pSO2 (oxygen saturation).

54. The method of claim 38, further comprising creating PPG signals from said detected optical data by calculating an rPPG trace signal using said time series;
estimating mean pulse rate from rPPG trace signals; calculating a mean instantaneous pulse rate; determining a rPPG signal according to PM (projection matrix) applying adaptive Wiener filtering and with an initial signal determined according to said instantaneous pulse rate frequency.

55. The method of claim 54, wherein said mean pulse rate is estimated using a match filter between two rPPG different analytic signals constructed from raw interpolated data (CHROM like and Projection Matrix (PM)).

56. The method of claim 55, wherein a cross-correlation is calculated to determine said mean instantaneous pulse rate, wherein frequency estimation is calculated according to non-linear least square (NLS) spectral decomposition.

57. The method of claim 56, wherein said PPG signals are further determined by applying an additional filter in the frequency domain to force signal reconstruction and an exponential filter applied on instantaneous RR values.

58. A system for obtaining a physiological signal from a subject, the system comprising: a camera for obtaining optical data from a face of the subject, a user computational device for receiving optical data from said camera, wherein said user computational device comprises a processor and a memory for storing a plurality of instructions, wherein said processor executes said instructions for analyzing the optical data to select data related to the face of the subject, detecting optical data from a skin of the face, determining a time series from the optical data by collecting the optical data until an elapsed period of time has been reached and then calculating the time series from the collected optical data for the elapsed period of time; and calculating the physiological signal from the time series; wherein said detecting said optical data from said skin of the face comprises determining a plurality of face boundaries, selecting the face boundary with the highest probability and applying a histogram analysis to video data from the face; wherein said histogram analysis comprises a histogram based classifier with a soft thresholding mechanism.

59. The system of claim 58, wherein said memory is configured for storing a defined native instruction set of codes and said processor is configured to perform a defined set of basic operations in response to receiving a corresponding basic instruction selected from the defined native instruction set of codes stored in said memory; wherein said memory stores a first set of machine codes selected from the native instruction set for analyzing the optical data to select data related to the face of the subject, a second set of machine codes selected from the native instruction set for detecting optical data from a skin of the face, a third set of machine codes selected from the native instruction set for determining a time series from the optical data by collecting the optical data until an elapsed period of time has been reached and then calculating the time series from the collected optical data for the elapsed period of time; and a fourth set of machine codes selected from the native instruction set for calculating the physiological signal from the time series.

60. The system of claim 59, wherein said determining said plurality of face boundaries comprises applying a multi-parameter convolutional neural net (CNN) to said video data to determine said face boundaries, such that said memory further comprises an eighth set of machine codes selected from the native instruction set for applying a multi-parameter convolutional neural net (CNN) to said video data to determine said face boundaries.

61. The system of claim 60, wherein said camera comprises a mobile phone camera and wherein said optical data is obtained as video data from said mobile phone camera.

62. The system of claim 61, wherein said computational device comprises a mobile communication device.

63. The system of claim 62, wherein said mobile phone camera comprises a rear facing camera and a fingertip of the subject is placed on said camera for obtaining said video data.

64. The system of claim 63, further comprising a flash associated with said mobile phone camera to provide light for obtaining said optical data.

65. The system of claim 64, wherein said memory further comprises a ninth set of machine codes selected from the native instruction set for determining a plurality of face or fingertip boundaries, a tenth set of machine codes selected from the native instruction set for selecting the face or fingertip boundary with the highest probability, and an eleventh set of machine codes selected from the native instruction set for applying a histogram analysis to video data from the face or fingertip.

66. The system of claim 65, further comprising combining analyzed data from images of the face and fingertip to determine the physiological measurement according to said instructions executed by said processor.

67. The system of claim 66, further comprising a display for displaying the physiological measurement and/or signal; wherein said user computational device further comprises said display; and wherein said user computational device further comprises a transmitter for transmitting said physiological measurement and/or signal.

68. A system for obtaining a physiological signal from a subject, the system comprising: a rear facing camera for obtaining optical data from a finger of the subject, a user computational device for receiving optical data from said camera, wherein said user computational device comprises a processor and a memory for storing a plurality of instructions, wherein said processor executes said instructions for analyzing the optical data to select data related to the face of the subject, detecting optical data from a skin of the finger, determining a time series from the optical data by collecting the optical data until an elapsed period of time has been reached and then calculating the time series from the collected optical data for the elapsed period of time; and calculating the physiological signal from the time series; wherein said detecting said optical data from said skin of the face comprises determining a plurality of face boundaries, selecting the face boundary with the highest probability and applying a histogram analysis to video data from the face; wherein said histogram analysis comprises a histogram based classifier with a soft thresholding mechanism.
